(19) United States
(12) Patent Application Publication
(10) Pub. No.: US 2026/0242310 A1
(43) Pub. Date: Aug. 20, 2026

(54) IMPROVED METHOD FOR DEHYDROGENATING HYDROCARBONS

(71) Applicant: ARKEMA FRANCE, Colombes (FR)

(72) Inventors: Jean-Luc DUBOS, Lacq (FR); Svetan KOLITCHEFF, Lacq (FR)

(73) Assignee: ARKEMA FRANCE, Colombes (FR)

(21) Appl. No.: 18/862,068

(22) PCT Filed: May 5, 2023
(86) PCT No.: PCT/FR2023/000083

§ 371 Date: Oct. 31, 2024

(30) Foreign Application Priority Data
May 10, 2022 (FR) 2204397

(51) Int. Cl. C07C 5/32; B01J 23/42; B01J 31/02; B01J 31/12

(52) U.S. Cl. CPC C07C 5/321; B01J 23/42; B01J 31/022; B01J 31/122; B01J 2231/766; B01J 2531/26; C07C 2527/02; C07C 2531/12

(57) ABSTRACT

The present invention relates to a process for the dehydrogenation of a hydrocarbon chain, in at least one reactor in the presence of a catalyst;
said process comprising a stage of introduction of a metal M upstream or at the inlet of the reactor(s), and
said metal M being chosen from the group consisting of zinc (Zn), tin (Sn), indium (In), zirconium (Zr), cerium (Ce), germanium (Ge), gallium (Ga), lead (Pb) and thallium (Tl), preferably zinc.

The present invention relates to an improved process for the dehydrogenation of hydrocarbon chains. The present invention also relates to a composition which makes possible in particular the use of such a process.

The dehydrogenation of hydrocarbon chains, and in particular of alkanes, is of great industrial interest. This is because there exists a growing demand for dehydrogenated hydrocarbon chains (hereinafter referred to as alkenes or olefins), which are essential synthesis intermediates for the chemical industry.

Thus, the conversion of propane into propylene and of isobutane into isobutene is of interest very particularly to manufacturers. Propylene is mainly polymerized to give polypropylene (PP), a staple polymer. It is used, for example, for the manufacture of molded parts in the automotive field, food packagings, furniture fabrics, disposable workwear or as an adjuvant to concrete. Isobutene can for its part be used to produce methyl tert-butyl ether or be polymerized to give polyisobutene (PIB). This polymer is used for the manufacture of inner tubes or of tubeless tire liners. Its secondary applications are highly varied: manufacture of chewing gums, sealants, vibration damping mastics, adhesives, coatings, cosmetics, insulating oils, and others. The catalytic dehydrogenation of hydrocarbon chains has been known for a long time. However, to date, the yields of these catalytic processes are between 50% and 85%. In view of the growing demand for olefins, it is desirable to improve these yields.

There thus exists a need to improve the existing dehydrogenation processes and in particular to improve their yields. There exists a need for dehydrogenation processes which are more efficient, more economical and easy to implement.

An objective of the present invention is to provide an improved process for the dehydrogenation of hydrocarbon chains, preferably with an improved yield.

Another objective of the present invention is to provide an improved dehydrogenation process which is easy to implement, and in particular which does not involve any modification of the pre-existing industrial installations.

An objective of the present invention is to provide a composition which is easy to use by the operators of the process, making it possible to improve the dehydrogenation reaction while being compatible with industrial installations.

The invention responds, in whole or in part, to the above objectives.

The present inventors have discovered that the introduction, upstream or at the inlet of the dehydrogenation reactor, of at least one metal M as defined below improves the yield of the reaction and/or increases the lifetime of the catalyst. In particular, the introduction of the metal M according to the invention makes it possible to obtain an increase in yield of between 1% and 10%, preferably between 2% and 5%. The addition of the metal M can make possible a controlled improvement in the yield, in particular by virtue of a specific and suitable formulation of said metal M.

Without wishing to be committed to a theory, the introduction of metal M makes it possible to generate catalytic sites which are more active on the dehydrogenation catalyst. Moreover, when the catalyst comprises several metals, the organization of the metal catalytic sites can prove to be difficult to control. Consequently, the addition of metal M according to the invention makes it possible to improve and/or to control the activity of the catalyst. The invention can also make possible the use and the handling of less complex and less toxic catalysts, which will possibly be easier to regenerate.

Adding a metal M separated from the catalyst, and optionally continuously, can also make it possible to combat its gradual deactivation, a problem well known to manufacturers in this type of process.

The inventors have also discovered that, in the form of an organometallic compound as defined below, said metal M improves the yield of the reaction while being perfectly adapted to the process and to the installations in which it is employed. This is because the organometallic compounds according to the invention can be dissolved in whole or in part of the reactant (that is to say, in the hydrocarbon chain to be dehydrogenated) and/or be dissolved in other additives introduced into the process.

In a very particularly preferred and surprising way, these organometallic compounds can be dissolved in compositions limiting or preventing the formation of coke (in particular on the catalyst), while retaining the gain in yield. Such compositions generally comprise sulfur compounds which will make it possible to passivate the metallurgy of the reactor and/or to selectively poison the catalyst in order to limit or prevent the formation of coke.

Thus, the organometallic compounds such as according to the invention can be dissolved in compositions comprising sulfur compounds as defined below. Alternatively, it is possible to speak of sulfur compound additivated with an organometallic compound. For example, dimethyl disulfide (hereinafter referred to as DMDS) additivated with an organometallic compound is spoken of.

These compositions exhibit the advantage of being able to be introduced into the process at the point(s) of introduction of additive(s) already in place in the dehydrogenation unit and making possible a controlled metering of the amount of metal M introduced. Thus, no modification of the installations is necessary in order to improve the dehydrogenation process, which is of great advantage to manufacturers.

### BRIEF DESCRIPTION OF THE INVENTION

It is specified that the expressions “from . . . to . . . ” and “of between . . . and . . . ” used in the present description should be understood as including each of the limits mentioned.

It is also specified that the ppm values express a fraction by weight, unless expressly mentioned otherwise.

The present invention relates to a process for the preparation of an olefin, which can also be referred to as dehydrogenation process. Said process comprises a stage of dehydrogenation of a hydrocarbon chain (referred to as stage b) below), in at least one reactor in the presence of a catalyst;
said process comprising a stage of introduction (referred to as stage a) below) of a metal M upstream or at the inlet of the reactor(s), and
said metal M being chosen from the group consisting of zinc (Zn), tin (Sn), indium (In), zirconium (Zr), cerium (Ce), germanium (Ge), gallium (Ga), lead (Pb) and thallium (Tl), preferably zinc.

The present invention also relates to a composition comprising:
a sulfur compound of following general formula (I):

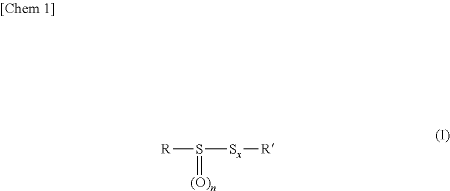

in which:
   R is chosen from a linear or branched alkyl radical containing from 1 to 4 carbon atoms and a linear or branched alkenyl radical containing from 2 to 4 carbon atoms,
   n is an integer equal to 0, 1 or 2,
   x is an integer chosen from 0, 1, 2, 3 or 4, and
   R′ is chosen from a linear or branched alkyl radical containing from 1 to 4 carbon atoms, a linear or branched alkenyl radical containing from 2 to 4 carbon atoms and, only when n=x=0, a hydrogen atom, and
a metal M chosen from the group consisting of zinc (Zn), tin (Sn), indium (In), zirconium (Zr), cerium (Ce), germanium (Ge), gallium (Ga), lead (Pb) and thallium (Tl), preferably zinc.

The present invention also relates to the use of said metal M in a process for the dehydrogenation of a hydrocarbon chain, preferably to increase the yield thereof.

The present invention also relates to the use of said composition in a process for the dehydrogenation of a hydrocarbon chain, preferably to increase the yield thereof.

Preferably, said metal M is in the form of an organometallic compound of general formula (II) as defined below.

### DETAILED DESCRIPTION OF THE INVENTION

The reaction for the dehydrogenation of hydrocarbon chains is a widely known reaction (cf. WO 2020/081421 and EP 3 240 770, for example). This reaction makes it possible to form a C═C double bond and in particular to obtain monoolefins from alkanes and/or diolefins from monoolefins. Preferably, alkanes are used to obtain monoolefins.

The term “hydrocarbon chain” is understood to mean in particular an alkane or an olefin. It can have between 2 and 30 carbon atoms, preferably between 2 and 10 carbon atoms.

The term “alkane” is understood to mean in particular a saturated, linear, branched or cyclic, preferably linear, hydrocarbon chain.

The term “olefin” is understood to mean in particular an unsaturated hydrocarbon chain, which can be a linear, branched or cyclic, preferably linear, mono- or diolefin.

The term “monoolefin” is understood to mean an unsaturated hydrocarbon chain comprising a single C═C double bond. The term “diolefin” is understood to mean an unsaturated hydrocarbon chain comprising two C═C double bonds.

The alkanes to be dehydrogenated can contain between 2 and 30 carbon atoms, for example between 2 and 10 carbon atoms, preferably between 3 and 6 carbon atoms. Propane, butane, isobutane and their mixtures are preferred, and entirely preferably propane, isobutane and their mixtures.

The monoolefins to be dehydrogenated can contain between 3 and 30 carbon atoms, for example between 3 and 10 carbon atoms, preferably between 3 and 6 carbon atoms. Mention may be made of but-1-ene.

Said hydrocarbon chains can be in liquid or gaseous form or as a gas-liquid mixture. They can be introduced as a mixture with a diluent into the dehydrogenation reactor. Said mixing with the diluent can be carried out upstream, at the inlet or in the reactor. Mention may in particular be made, as diluent, of hydrogen, steam, methane, ethane, carbon dioxide, nitrogen, argon, or any other inert gas for the dehydrogenation reaction, and their mixtures. Preferably, hydrogen and steam are used. A molar ratio (diluent/hydrocarbon chain) of between 0.1:1 and 40:1, preferably between 0.4:1 and 10:1, is generally used.

The hydrogenation reaction can be carried out at a temperature of between 400° C. and 900° C., preferably between 520° C. and 650° C.

The hydrogenation reaction can be carried out at a pressure of between 0.01 and 10 bars, preferably between 1 and 6 bars, more preferentially between 1 and 3 bars.

The catalyst used is a dehydrogenation catalyst, preferably a heterogeneous dehydrogenation catalyst. In particular, it comprises a supported metal.

More particularly, it comprises at least one metal chosen from groups 6, 8, 9 and 10 of the Periodic Table of the Elements (formerly groups VIA and VIII). Preferably, the catalyst comprises a metal chosen from chromium, platinum or palladium, more preferentially platinum.

The chromium, the platinum or the palladium present can be the predominant metal in the catalyst (by weight, with respect to all the metals contained in the catalyst). The catalyst can also comprise other metals, such as rhenium, germanium or tin, for example one, two or three metals. The catalyst can also comprise a promoter, preferably a metal chosen from alkali metals or alkaline earth metals, and preferentially potassium.

According to one embodiment, said catalyst before stage a) of introduction of the metal M comprises less than 5% by weight of said metal M, preferably less than 1% by weight, with respect to the total weight of the catalyst. In particular, the catalyst before stage a) of introduction of the metal M does not comprise zinc.

The metal(s) of the above catalyst can be present as element(s) or as compound(s), for example in an oxidized or reduced form. Such catalysts are known to a person skilled in the art and are described, for example, in the document U.S. Pat. No. 3,723,557.

The support of the catalyst can be of any type known to a person skilled in the art and preferably chosen from porous supports. For example, it can be chosen from silica, alumina, silica-alumina, molecular sieves, titanium dioxide and zirconia, preferably alumina.

A particularly preferred catalyst is a catalyst comprising platinum supported on an alumina, preferably a catalyst consisting essentially of platinum supported on an alumina.

Such catalysts can undergo an activation, for example a reduction and/or sulfidation, in situ or ex situ, prior to their use in the dehydrogenation reaction. This operation is known to a person skilled in the art and can be carried out according to conventional techniques.

The reaction can be carried out in one or more reactors. Said reactors can be placed in parallel or in series. The reactors can be fixed, moving or fluidized catalytic bed reactors. Use is preferentially made of moving catalytic bed reactors in series.

In the presence of several reactors, heating means between each reactor can be installed, so as to ensure that the temperature of entry of the reactants into the reactors is the desired reaction temperature. In this case, the metal M can be introduced before or after the heating means, preferably before.

After the dehydrogenation reaction, the process according to the invention can comprise other stages up to the recovery of the olefin and of the hydrogen (H₂) produced. They make it possible in particular to obtain an olefin-enriched stream (stream A and D below), which can be subsequently purified, if necessary.

The stream S exiting the reactor can comprise hydrogen, olefin and possibly unreacted hydrocarbon chain. When the reactor is a moving catalytic bed reactor, the heterogeneous catalyst can also be recovered at the reactor outlet separately from the exiting stream S.

Once the exiting stream S has been recovered, its separation can be carried out by any technique known to a person skilled in the art. It can, for example, be cooled and then condensed, so as to obtain:
a gas stream H comprising hydrogen; and
a stream A, which is preferably liquid, comprising the olefin and possibly the unreacted hydrocarbon chain.

The stream A can subsequently be recovered and separated, so as to obtain:
a liquid stream D comprising the olefin; and
a liquid stream E comprising the unreacted hydrocarbon chain.

The streams H and/or E can be recycled, in whole or in part, to the reaction stage. In the presence of several reactors, they can be reintroduced into one, several or all of the reactors.

The liquid stream D is recovered and then optionally purified, if necessary.

The dehydrogenation process can also comprise a stage of regeneration of the catalyst (referred to as stage e)). This is because the catalyst can gradually become deactivated, in particular due to the formation of coke on the latter. A regeneration stage may thus prove to be necessary in order to restore the whole or part of its activity. This regeneration can be carried out according to conventional techniques.

For example, the catalyst can be regenerated by incinerating the coke (at at least 600° C.) and then carrying out an oxychlorination in order to redisperse the catalytic metal. The oxychlorination is generally carried out by bringing the catalyst into contact with a gas comprising a halogen, preferably chlorine, at high temperatures, for example between 500° C. and 550° C.

Following the regeneration stage, the catalyst can be reduced by bringing into contact with hydrogen, so as to reduce the metals oxidized during the regeneration. The regenerated and optionally reduced catalyst can be recycled to the reaction stage, alone or as a mixture with fresh catalyst.

According to one embodiment, the process according to the invention comprises at least the following stages:
a) introduction of said metal M upstream or at the inlet of the reactor(s);
b) dehydrogenation reaction of the hydrocarbon chain in the moving catalytic bed reactor(s), so as to obtain an exiting stream S comprising an olefin and hydrogen;
c) recovery and separation of the exiting stream S resulting from stage b), so as to obtain:
   a stream A, which is preferably liquid, comprising the olefin, and
   a gaseous stream H comprising hydrogen;
d) optional recovery of the catalyst;
e) optional regeneration of said catalyst;
f) optional recycling to stage b) of the regenerated catalyst.

More particularly, the process according to the invention comprises at least the following stages:
a) introduction of said metal M upstream or at the inlet of the reactor(s);
b) dehydrogenation reaction of an alkane in the moving catalytic bed reactor(s), so as to obtain an exiting stream S comprising an olefin, the unreacted alkane and hydrogen;
c) recovery and separation of the exiting stream S resulting from stage b), so as to obtain:
   a stream A, which is preferably liquid, comprising the olefin and the unreacted alkane, and
   a gaseous stream H comprising hydrogen;
d) optional recovery of the catalyst;
e) optional regeneration of said catalyst;
f) optional recycling to stage b) of the regenerated catalyst;
g) recovery and separation of the stream A to obtain:
   a stream D comprising the olefin; and
   a stream E comprising the unreacted alkane;
h) optional recycling of the stream E to stage b).

As indicated above, the stream H and/or E can be recycled, in whole or in part, to the reaction stage. In the presence of several reactors, it can be reintroduced into one, several or all of the reactors.

The process according to the invention can be carried out batchwise or continuously, preferably continuously. Several dehydrogenation processes are thus known and mention may be made of those mentioned in the publication “Propylene Production by Propane Dehydrogenation (PDH) (Process Review)” by Amir Razmi, 2019 (https://www.academia.edu/39604411/Propylene_Production_by_Propane_Dehydrogenation_PDH_Process_Review), such as the following processes: UOP-Honeywell “Oleflex™”, Lummus “Catofin@” and “Star process@” by ThyssenKrupp Uhde.

The metal M according to the invention is chosen from the group consisting of zinc (Zn), tin (Sn), indium (In), zirconium (Zr), cerium (Ce), germanium (Ge), gallium (Ga), lead (Pb) and thallium (Tl). In particular, the metal M is chosen from the group consisting of zinc (Zn), tin (Sn), lead (Pb) and indium (In). Entirely preferably, the metal M is zinc (Zn).

Said metal M is more particularly in the form of an organometallic compound, preferably of following general formula (II):

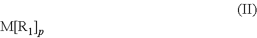

in which:
M is the metal as defined above;
p is an integer of between 1 and 6, preferably between 2 and 4; and
the R₁ radicals, which are identical or different, represent, independently of one another, a hydrocarbon radical containing from 1 to 12 carbon atoms.

Preferably, the R₁ radicals, which are identical or different, are chosen, independently of one another, from the group consisting of:
a linear, branched or cyclic alkyl containing between 1 and 10 carbon atoms;
a linear, branched or cyclic alkenyl containing between 1 and 10 carbon atoms;
an aryl containing between 6 and 10 carbon atoms, said aryl optionally being substituted by one or more linear or branched alkyl groups containing from 1 to 10 carbon atoms; and an alkylaryl containing between 6 and 12 carbon atoms.

In particular, the R₁ radicals, which are identical or different, are chosen, independently of one another, from the group consisting of:
a linear, branched or cyclic alkyl containing between 1 and 10 carbon atoms; and
an aryl containing between 6 and 10 carbon atoms, said aryl optionally being substituted by one or more linear or branched alkyl groups containing from 1 to 10 carbon atoms.

In particular, the R₁ radicals, which are identical or different, are chosen, independently of one another, from linear, branched or cyclic alkyls containing between 1 and 10 carbon atoms.

Preferably, the R₁ radicals are identical. Preferably, said alkyls contain between 1 and 6 carbon atoms, more preferentially between 1 and 3 carbon atoms. For example, said alkyls are chosen from the group consisting of methyl, ethyl, n-propyl and isopropyl. For example, said aryl is a phenyl and/or said alkylaryl is benzyl and/or said alkenyl is cyclopentadienyl.

In particular, the organometallic compound is chosen from those of following formulae:

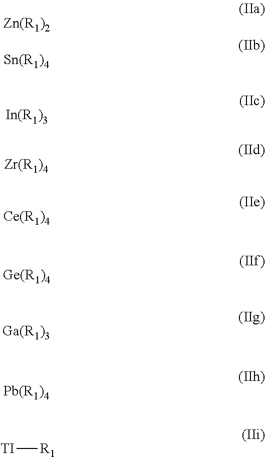

in which the R₁ radicals, which are identical or different, are as defined for the general formula (II).

In particular, the metal M is zinc and is in the form of an organometallic compound of following general formula (IIa):

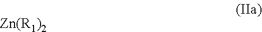

in which the R₁ radicals, which are identical or different, are as defined for the general formula (II).

Mention may be made, as organometallic compounds of general formula (II), of: tetramethyltin, trimethylindium, tetrabenzylzirconium, tetraethylgermanium, trimethylgallium, tetramethyllead, cyclopentadienylthallium, dimethylzinc (Zn(CH₃)₂), diethylzinc (Zn(C₂H₅)₂), diisopropylzinc (Zn(i-C₃H₇)₂), dipropylzinc (Zn(C₃H₇)₂) and diphenylzinc (Zn(C₆H₅)₂).

Said organometallic compound is in particular chosen from the group consisting of: Zn(CH₃)₂, Zn(C₂H₅)₂, Zn(i-C₃H₇)₂, Zn(C₃H₇)₂ and Zn(C₆H₅)₂. The CAS numbers for these compounds are as follows: Zn(CH₃)₂: 544-97-8; Zn(C₂H₅)₂: 577-20-0; Zn(C₃H₇)₂: 628-91-1; Zn(i-C₃H₇)₂: 625-81-0 and Zn(C₆H₅)₂: 1078-58-6.

More preferably, said metal compound is chosen from Zn(CH₃)₂, Zn(C₂H₅)₂ and Zn(C₃H₇)₂; more particularly Zn(CH₃)₂ and Zn(C₂H₅)₂.

Said organometallic compounds can be formulated in a solvent, especially an organic solvent, in particular chosen from toluene and alkanes containing between 5 and 10 carbon atoms.

Preferably, the solvent is chosen from toluene, heptane, hexane or their mixtures. The organometallic compounds as mentioned above are commercially available in the form of 1 M or 2M solutions.

The introduction of the metal M (stage a)), optionally in organometallic form as defined above, can be carried out by any known means, for example by injection.

It is understood that said metal M does not form part of the dehydrogenation catalyst during its introduction in stage a)).

In particular, said metal M is introduced separately from said dehydrogenation catalyst.

The introduction can be one-off, semi-continuous or continuous, preferably continuous. It can be carried out at the start-up of a unit for the production of olefins or during the whole or part of the production of olefins. The amounts of metal M which are introduced may or may not vary during the duration of the addition, for example with a gradient which decreases or increases during the duration of the addition.

It is understood that, in the case of reactors in series, the introduction of the metal M can take place upstream or at the inlet of the first reactor of the series and/or upstream or at the inlet of any one or of several of the following reactors. The introduction of the metal M can thus take place at different places in the installations of the process.

Said metal M, optionally in said organometallic form, is preferentially comprised in a composition also comprising a sulfur compound of following general formula (I):

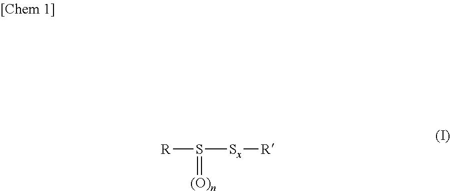

in which:
R is chosen from a linear or branched alkyl radical containing from 1 to 4 carbon atoms and a linear or branched alkenyl radical containing from 2 to 4 carbon atoms,
n is an integer equal to 0, 1 or 2,
x is an integer chosen from 0, 1, 2, 3 or 4, and
R′ is chosen from a linear or branched alkyl radical containing from 1 to 4 carbon atoms, a linear or branched alkenyl radical containing from 2 to 4 carbon atoms and, only when n=x=0, a hydrogen atom.

Preferably, R and R′, which are identical or different, are chosen from linear or branched alkyls containing from 1 to 4 carbon atoms. Preferably, R and R′ are identical.

In particular, n is equal to 0. In particular, x is chosen from 1, 2, 3 or 4, preferably 1 or 2 and more preferentially 1.

More particularly, the sulfur compound is of following general formula (Ia):

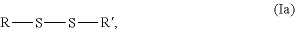

in which the R and R′ radicals are as defined above.

It is understood that mixtures of two or more sulfur compounds of general formula (I) can be used according to the present invention. In particular, mixtures of di- and/or polysulfides can be used, for example mixtures of disulfides, such as disulfide oils (DSO).

Preferably, the sulfur compound of general formula (I) is chosen from dimethyl disulfide (DMDS), dimethyl sulfide (DMS), dimethyl sulfoxide (DMSO) and di(tert-butyl) polysulfides. In particular, said sulfur compound of general formula (I) is chosen from dimethyl disulfide (DMDS), dimethyl sulfide (DMS) and di(tert-butyl) polysulfides, preferably dimethyl disulfide.

Entirely preferably, said sulfur compound is dimethyl disulfide. Said sulfur compound is in particular commercially available. Mention may be made of DMDS Evolution® E2 sold by Arkema.

Preference is very particularly given to the compositions comprising:
DMDS; and
an organometallic compound of following general formula (II):

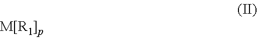

in which M, R₁ and p are as defined above.

In particular, said composition comprises at least 80%, preferably at least 95%, more preferentially at least 99%, by total weight, of sulfur compound(s), with respect to the total weight of the composition. It can contain one or more odor-masking agents (see, for example, the international application WO 2011/012815A1).

Said composition can also comprise a solvent, especially an organic solvent, in particular chosen from toluene and alkanes containing between 5 and 10 carbon atoms. Preferably, the solvent is chosen from toluene, heptane, hexane or their mixtures. Preferably, said composition comprises between 0.001% and 10%, for example between 0.005% and 1.5%, by weight, of said solvent, with respect to the total weight of the composition.

The amount of metal M is preferentially of between 1 and 10 000 ppm, preferably between 1 and 1000 ppm, for example between 1 and 500 ppm, more preferably between 5 and 200 ppm, with respect to the total weight of the sulfur compound(s) and of the metal M. It can be of between 5 and 100 ppm, more particularly between 10 and 100 ppm, with respect to the total weight of the sulfur compound and of the metal M.

The amount of the composition introduced (during stage a)) can be of between 5 and 200 ppm, preferably between 10 and 90 ppm, for example 25, 50 or 75 ppm, with respect to the weight of the hydrocarbon chain to be dehydrogenated.

Said metal M, optionally in organometallic form, or said composition can be mixed beforehand with the whole or part of the hydrocarbon chain to be dehydrogenated before it is introduced upstream or at the inlet of the reactor(s).

The compositions comprising a sulfur compound and a metal M as defined above are novel and also form part as such of the present invention.

Preference is very particularly given to the compositions comprising:
DMDS; and
an organometallic compound of following general formula (II):

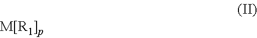

in which M, R₁ and p are as defined above.

Such compositions are particularly suitable for introduction into the process as such according to the invention. They are in particular liquid (at ambient temperature, for example between 5° C. and 40° C.). The organometallic compounds are definitely dissolved in DMDS, which makes possible great ease of handling and control of the amount of metal M introduced.

The compositions according to the invention can be prepared by simple mixing of said sulfur compound and of the metal M. Said metal M can be in organometallic form, optionally dissolved in a solvent as defined above.

They can also be in the form of a kit comprising the sulfur compound and the metal M in order to be formed upstream, at the inlet or in situ in the dehydrogenation reactor.

The present invention also relates to the use of said metal M, optionally in organometallic form, in a process for the dehydrogenation of a hydrocarbon chain to give an olefin, preferably to increase the yield thereof. It is understood that said metal M can be included in a composition as defined above.

### DESCRIPTION OF THE FIGURES

FIG. 1 represents the yield of the dehydrogenation reaction obtained as a function of time. DMDS alone is represented by the curve containing the squares, DMDS additivated with Zn(CH₃)₂ is represented by the continuous curve and DMDS additivated with Zn(C₂H₅)₂ is represented by the curve containing the diamonds.

### EXAMPLES

### Example 1: Process According to the Invention

The dehydrogenation of propane is carried out in a reactor at a temperature of 625° C., at a pressure of 3 bars and in the presence of a catalyst of Pt/Al₂O₃ type (Pt/Al₂O₃ catalyst from Strem Chemicals Inc.) on a fixed bed over a period of 20 h.

The propane flow rate is 2.2 Sl/h.

A continuous injection of 75 ppm of different DMDS compositions is carried out upstream of the reactor.

Compositions according to the invention were prepared by adding, to DMDS, 50 ppm of the following commercial solutions (supplier Aldrich):
a 1M solution of dimethylzinc,
a 1M solution of diethylzinc.

Clear solutions of additivated DMDS are obtained.

A comparative composition of DMDS not additivated with metal M is also tested.

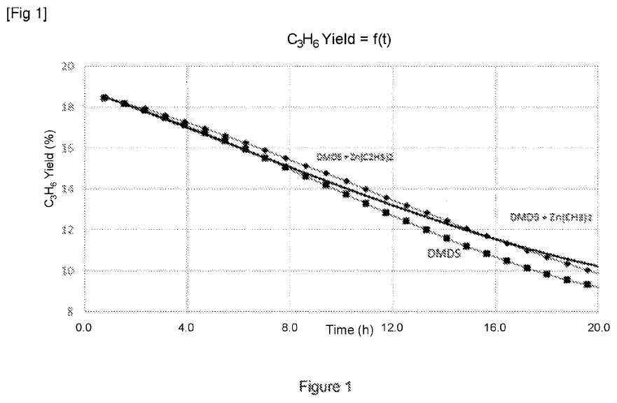

The results obtained are given by FIG. 1.

The cumulative amounts of propylene obtained at 20 h are as follows:
6.08 Sl with DMDS alone;
6.23 Sl with DMDS additivated with Zn(CH₃)₂, i.e. an increase in the final yield of 2.6%; and
6.37 Sl with DMDS additivated with Zn(C₂H₅)₂, i.e. an increase in the final yield of 4.8%.

The propylene yield is thus improved by virtue of the present invention.

1. A process for the preparation of an olefin comprising a stage of dehydrogenation of a hydrocarbon chain, in at least one reactor in the presence of a catalyst;
said process comprising a stage of introduction of a metal M upstream or at the inlet of the reactor(s), and
said metal M being chosen from the group consisting of zinc (Zn), tin (Sn), indium (In), zirconium (Zr), cerium (Ce), germanium (Ge), gallium (Ga), lead (Pb) and thallium (Tl), preferably zinc.

2. The process as claimed in claim 1, in which said metal M is in the form of an organometallic compound of following general formula (II): 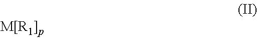
in which:
M is said metal;
p is an integer of between 1 and 6, preferably between 2 and 4; and
the R₁ radicals, which are identical or different, represent, independently of one another, a hydrocarbon radical containing from 1 to 12 carbon atoms.

3. The process as claimed in claim 2, in which said organometallic compound is chosen from the group consisting of: tetramethyltin, trimethylindium, tetrabenzylzirconium, tetraethylgermanium, trimethylgallium, tetramethyllead, cyclopentadienylthallium, dimethylzinc, diethylzinc, diisopropylzinc, dipropylzinc and diphenylzinc.

4. The process as claimed in claim 1, in which the metal M is comprised in a composition also comprising a sulfur compound of following general formula (I): 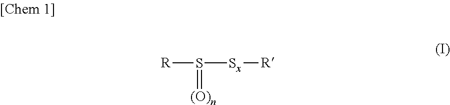
in which:
R is chosen from a linear or branched alkyl radical containing from 1 to 4 carbon atoms and a linear or branched alkenyl radical containing from 2 to 4 carbon atoms,
n is an integer equal to 0, 1 or 2,
x is an integer chosen from 0, 1, 2, 3 or 4, and
R′ is chosen from a linear or branched alkyl radical containing from 1 to 4 carbon atoms, a linear or branched alkenyl radical containing from 2 to 4 carbon atoms and, only when n=x=0, a hydrogen atom.

5. The process as claimed in claim 4, in which said sulfur compound of general formula (I) is chosen from dimethyl disulfide (DMDS), dimethyl sulfide (DMS) and di(tert-butyl) polysulfides, preferably dimethyl disulfide.

6. The process as claimed in claim 1, comprising at least the following stages:
a) introduction of the metal M upstream or at the inlet of the reactor(s);
b) dehydrogenation reaction of the hydrocarbon chain in the moving catalytic bed reactor(s), so as to obtain an exiting stream S comprising an olefin and hydrogen;
c) recovery and separation of the exiting stream S resulting from stage b), so as to obtain:
a stream A comprising the olefin; and
a stream H comprising hydrogen;
d) optional recovery of the catalyst;
e) optional regeneration of said catalyst; and
f) optional recycling to stage b) of the regenerated catalyst.

7. A composition comprising:
a sulfur compound of following general formula (I): 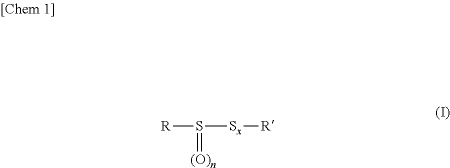
in which:
R is chosen from a linear or branched alkyl radical containing from 1 to 4 carbon atoms and a linear or branched alkenyl radical containing from 2 to 4 carbon atoms,
n is an integer equal to 0, 1 or 2,
x is an integer chosen from 0, 1, 2, 3 or 4, and
R′ is chosen from a linear or branched alkyl radical containing from 1 to 4 carbon atoms, a linear or branched alkenyl radical containing from 2 to 4 carbon atoms and, only when n=x=0, a hydrogen atom, and
a metal M chosen from the group consisting of zinc (Zn), tin (Sn), indium (In), zirconium (Zr), cerium (Ce), germanium (Ge), gallium (Ga), lead (Pb) and thallium (Tl), preferably zinc.

8. The composition as claimed in claim 7, in which said sulfur compound of general formula (I) is chosen from dimethyl disulfide (DMDS), dimethyl sulfide (DMS) and di(tert-butyl) polysulfides, preferably dimethyl disulfide.

9. The composition as claimed in claim 7, in which said metal M is in the form of an organometallic compound of following general formula (II): 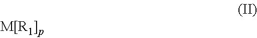
in which:
M is said metal;
p is an integer of between 1 and 6, preferably 2, 3 or 4; and
the R₁ radicals, which are identical or different, represent, independently of one another, a hydrocarbon radical comprising from 1 to 12 carbon atoms.

10. The composition as claimed in claim 7, also comprising an organic solvent.

11. The use of a composition as defined in claim 7, in a process for the dehydrogenation of a hydrocarbon chain to give an olefin.
